(19) United States
(12) Patent Application Publication
(10) Pub. No.: US 2022/0181026 A1
(43) Pub. Date: Jun. 9, 2022

(54) INFORMATION PROCESSING APPARATUS, INFORMATION PROCESSING SYSTEM, INFORMATION PROCESSING METHOD, AND STORAGE MEDIUM

(71) Applicant: CANON KABUSHIKI KAISHA, Tokyo (JP)

(72) Inventor: Yukiteru Masuda, Kanagawa (JP)

(21) Appl. No.: 17/538,929

(22) Filed: Nov. 30, 2021

(30) Foreign Application Priority Data
Dec. 4, 2020 (JP) 2020-201484

(51) Int. Cl. G16H 50/20; G06F 3/14; G06N 5/04

(52) U.S. Cl. CPC G16H 50/20; G06N 5/04; G06F 3/14

(57) ABSTRACT

An information processing apparatus includes an inference process package acquisition unit, a determination unit, and a setting unit. The inference process package acquisition unit acquires an inference process package that is applied to medical data and includes a single or a plurality of processes including an inference process. The determination unit determine, in a case where a plurality of the inference process packages is acquired, a process common to greater than or equal to two inference process packages among processes included in the plurality of the inference process packages. The setting unit sets a procedure of the plurality of processes including the common process to be applied to the medical data based on a determination result of the determination unit.

### BACKGROUND

### Field

The present disclosure relates to an information processing apparatus that determines a process common to a plurality of inference process packages to be applied to medical data, an information processing method, and a non-transitory storage medium.

### Description of the Related Art

In the medical field, doctors diagnose patients using image data of the insides of the patients' bodies captured by image diagnostic apparatuses, such as an X-ray computed tomography (CT) apparatus, a magnetic resonance imaging (MRI) apparatus, or an ultrasonic diagnostic apparatus, and medical data, such as electrocardiogram information obtained by an electrocardiograph.

In order to support diagnoses by doctors, various methods are have been developed for executing inference processes (e.g., detecting a pulmonary nodule from a chest CT image) regarding medical data using inference models obtained by machine learning.

A method has also been developed in which a plurality of inference processes is executed on single medical data using such a plurality of inference models. For example, Japanese Patent Application Laid-Open No. 2019-191772 discusses a method in which each of a plurality of inference models is applied to single medical image data to infer a plurality of image findings (e.g., a calcification concentration ratio, and arteriovenous involvement in a pulmonary nodule).

According to Japanese Patent Application Laid-Open No. 2019-191772, a process common to a plurality of inference process packages which include a plurality of processes including an inference process is set in advance, in order to reduce the number of processing times necessary to derive the image findings. However, in a case where a combination of the plurality of inference processes to be applied is changed according to medical data, it takes time and effort to set the common process in advance with respect to a plurality of inference process packages which include a plurality of processes including an inference process.

### SUMMARY

Various embodiments of the present disclosure are directed to the provision of an information processing apparatus, an information processing method, a storage medium, and an information processing system that can determine a process common to a plurality of inference process packages to be applied to medical data.

According to one embodiment of the present disclosure, an information processing apparatus includes an inference process package acquisition unit, a determination unit, and a setting unit. The inference process package acquisition unit acquires an inference process package that is applied to medical data and includes a single or a plurality of processes including an inference process. The determination unit determine, in a case where a plurality of the inference process packages is acquired, a process common to greater than or equal to two inference process packages among processes included in the plurality of the inference process packages. The setting unit sets a procedure of the plurality of processes including the common process to be applied to the medical data based on a determination result of the determination unit.

Further features of the present disclosure will become apparent from the following description of example embodiments with reference to the attached drawings.

### BRIEF DESCRIPTION OF THE DRAWINGS

FIG. 1 illustrates a functional configuration of an information processing system according to a first example embodiment.

FIG. 2 illustrates a configuration of a computer in an information processing apparatus according to the first example embodiment.

FIG. 3 is a flowchart illustrating a processing procedure of the information processing apparatus according to the first example embodiment.

FIGS. 4A to 4C illustrate specific examples of processes included in a plurality of inference process packages according to the first example embodiment.

FIG. 5 is a flowchart illustrating a processing procedure of an information processing apparatus according to a second example embodiment.

FIG. 6 illustrates a functional configuration of an information processing system according to a third example embodiment.

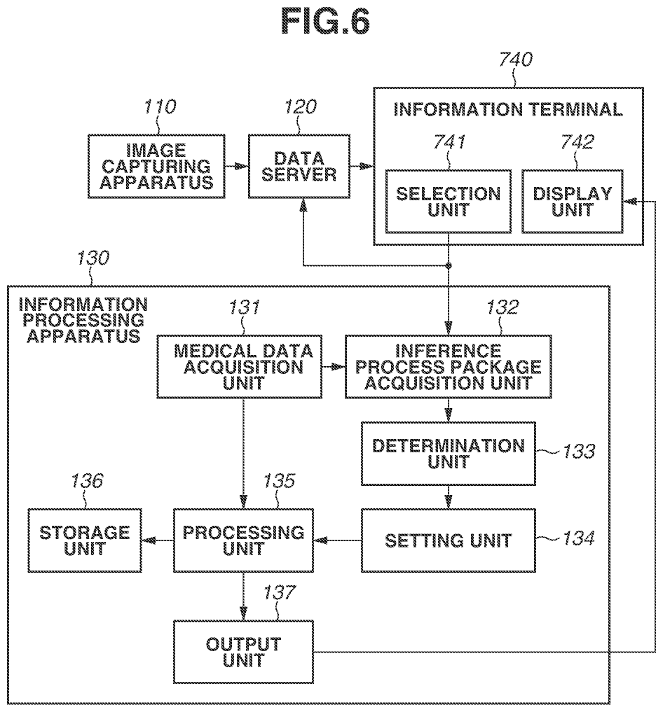

### DESCRIPTION OF THE EMBODIMENTS

Example embodiments according to the present disclosure will be described below with reference to the attached drawings. The same component is denoted with the same reference number, and the description thereof is omitted. However, the scope of the present invention is not limited to the example embodiments described below.

An information processing system according to a first example embodiment is described below. According to the present example embodiment, an information processing apparatus 130 included in the information processing system determines a process common to a plurality of inference process packages to be applied to acquired medical data, sets an appropriate processing procedure based on the determination result, and executes a process to the medical data based on the set processing procedure.

A case in which chest computed tomography (CT) image data obtained by imaging a subject by an X-ray CT apparatus is used as medical data of a process target is described below as an example. An image capturing apparatus that performs imaging and obtains image data is not limited to an X-ray CT apparatus and may be a magnetic resonance imaging (MRI) apparatus or an ultrasonic diagnostic apparatus. Further, medical data is not limited to image data, and may include character data and audio data.

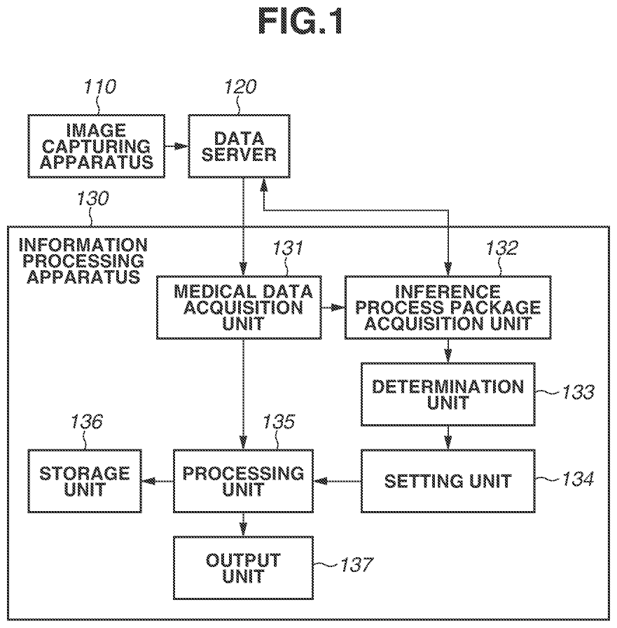

FIG. 1 is a block diagram illustrating a configuration example of the information processing system according to the present example embodiment. The information processing system includes an image capturing apparatus (an X-ray CT apparatus) 110, a data server 120, and the information processing apparatus 130. Each configuration in the information processing system is connected to each other in a communicable manner by, for example, a network.

The information processing system stores medical data (chest CT image data) captured by the image capturing apparatus 110 in the data server 120. The data server 120 stores a plurality of inference process packages which is described below in addition to the medical data captured by the image capturing apparatus 110. Further, the data server 120 may store processed medical data stored in a storage unit 136 included in the information processing apparatus 130 described below. In data management by the data server 120, for example, a system such as a picture archiving and communication system (PACS) may be used which stores and manages medical data such as image data via the network.

The information processing apparatus 130 includes a medical data acquisition unit 131, an inference process package acquisition unit 132, a determination unit 133, and a setting unit 134. The information processing apparatus 130 further includes a processing unit 135, the storage unit 136, and an output unit 137.

The information processing apparatus 130 acquires, from the data server 120, the medical data captured by the image capturing apparatus 110 using the medical data acquisition unit 131.

The inference process package acquisition unit 132 acquires an inference process package which includes a single or a plurality of processes including an inference process. The inference process package is applied to the medical data acquired by the medical data acquisition unit 131. The inference process package further includes, for example, a process accompanying the inference in addition to the inference process. In a case where a plurality of inference process packages is acquired, the determination unit 133 determines a process common to two or more inference process packages among processes included in the plurality of inference process packages.

The setting unit 134 sets a procedure of a plurality of processes including the common process to be applied to the medical data based on the determination result of the determination unit 133. The information processing apparatus 130 is configured as described above, thus can determine the process common to the plurality of inference process packages with respect to the medical data, and set the procedure for efficiently executing a plurality of inference processes.

The information processing apparatus 130 further includes the processing unit 135 which executes the process based on the processing procedure set by the setting unit 134. The storage unit 136 stores a processing result of the processing unit 135. The storage unit 136 may cause the data server 120 to store the processing result. The information processing apparatus 130 further includes the output unit 137 that outputs the processing result of the processing unit 135. With the present configuration, the processing unit 135 can execute the process based on the processing procedure set by the setting unit 134, the storage unit 136 can store the processing result, and the output unit 137 can output the processing result. The information processing apparatus 130 can process the medical data at an appropriate number of processing times and comprehend the processing result of the process, accordingly. According to the present example embodiment, it is described that a function of each unit is realized by the information processing apparatus 130. However, each unit included in the information processing apparatus 130 may function as an information processing system that is realized by a plurality of apparatuses.

Each unit in the information processing apparatus 130 according to the present example embodiment is described in detail below.

### (Medical Data Acquisition Unit)

The medical data acquisition unit 131 acquires medical data from the data server 120 in response to a user instruction. The medical data may be any of audio data, waveform data, and text data as well as image data acquired by the image capturing apparatus 110. The medical data acquisition unit 131 may be realized by a different apparatus, and the medical data acquired by the different apparatus may be transmitted to the information processing apparatus 130.

### (Inference Process Package Acquisition Unit)

The inference process package acquisition unit 132 acquires, from the data server 120, the plurality of inference process packages to be applied to the medical data acquired by the medical data acquisition unit 131. Acquisition of the inference process package may be executed in response to a user instruction or may be automatically executed when the medical data is acquired by the medical data acquisition unit 131.

The inference process package includes a single or a plurality of processes which includes the inference process using an inference model. The processes included in the inference process package may be a preprocess to be applied to the medical data in order to cause the inference model to execute the inference process and a post-process to be applied to an inference result of the inference process, in addition to the inference process. Further, a process other than the preprocess and the post-process may be included as a plurality of processes included in the inference process package.

### (Determination Unit)

In a case where the plurality of inference process packages is acquired by the inference process package acquisition unit 132, the determination unit 133 determines a common process among the processes included in each of the plurality of inference process packages. Determination of the common process performed by the determination unit 133 will be described in detail below with reference to the attached drawings.

### (Setting Unit)

The setting unit 134 sets the processing procedure to be applied to the medical data based on the determination result of the determination unit 133. Regarding the processing procedure to be applied to the medical data, the setting unit 134 sets the processing procedure such that the process common to the plurality of inference process packages is executed in priority.

### (Processing Unit)

The processing unit 135 executes the process to the medical data according to the processing procedure set by the setting unit 134. In this case, the process determined as the process common to the plurality of inference process packages is executed only once, and the result is shared by next processes in the plurality of inference process packages.

### (Storage Unit)

The storage unit 136 stores the processing result of the processing unit 135 in the data server 120. Further, the storage unit 136 may store the processing result in a storage device, such as a hard disk provided in the information processing apparatus 130.

### (Output Unit)

The output unit 137 outputs the processing result executed to the medical data by the processing unit 135. The output by the output unit 137 may be displayed on a not-illustrated display unit, such as a display provided in the information processing apparatus 130, or the processing result may be output to an external apparatus.

Arithmetic circuits used in the medical data acquisition unit 131, the inference process package acquisition unit 132, the determination unit 133, the setting unit 134, the processing unit 135, the storage unit 136, and the output unit 137 may be specially designed processors. Each of these arithmetic circuits may be configured with different types of hardware. Further, at least a part of the arithmetic circuit may be configured with single hardware. In other words, the medical data acquisition unit 131, the inference process package acquisition unit 132, the determination unit 133, the setting unit 134, the processing unit 135, the storage unit 136, and the output unit 137 can be configured with a processor, such as a central processing unit (CPU) and a graphics processing unit (GPU), and an arithmetic circuit, such as a field programmable gate array (FPGA) chip. These units may be configured with not only a single processor and arithmetic circuit but also a plurality of processors and arithmetic circuits.

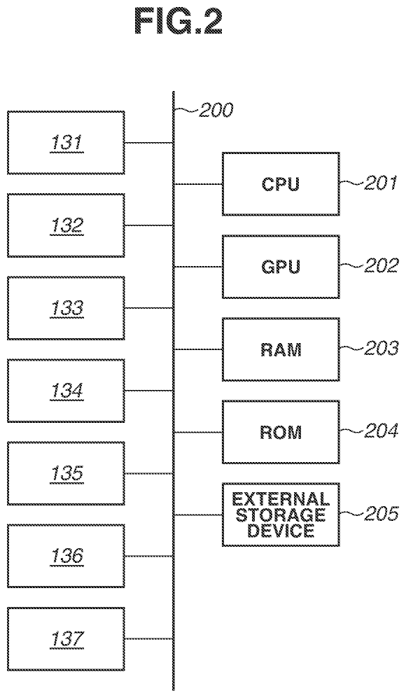

FIG. 2 illustrates a specific configuration example of the arithmetic circuits used in the following units: the medical data acquisition unit 131, the inference process package acquisition unit 132, the determination unit 133, the setting unit 134, the processing unit 135, the storage unit 136, and the output unit 137. The arithmetic circuit used in each unit included in the information processing apparatus 130 is configured with a CPU 201, a GPU 202, a random access memory (RAM) 203, a read-only memory (ROM) 204, and an external storage device 205, which are connected to each other by a system bus 200. The information processing apparatus 130 may also be connected to a mouse and a keyboard (not illustrated) serving as input interfaces and a display (not illustrated) serving as an output interface.

A function of each unit included in the information processing apparatus 130 may be formed as an on-premise system and execute the process. Alternatively, a part of the function may exist as a program in a server or the like in the network or in a cloud and execute the process. The medical data may also be acquired from an information terminal or the like which is separately held by a user, and the processing result may be output to the information terminal.

Each unit in the information processing apparatus 130 may be configured as a separate apparatus or may be configured as an information processing system including an apparatus in which at least a part of the configuration of the information processing apparatus 130 is integrated.

### (Determination Flow)

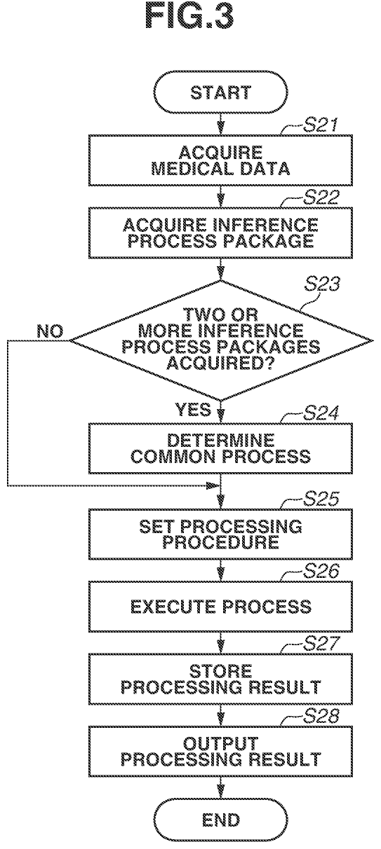

Processes executed by the information processing apparatus 130 according to the present example embodiment will now be described with reference to a flowchart in FIG. 3. The information processing apparatus 130 starts the process in the flowchart in FIG. 3 upon receiving an instruction from a user.

In step S21, the medical data acquisition unit 131 communicates with the data server 120, acquires a list of the medical data stored in the data server 120, and presents the list to the user via a not-illustrated interface. The medical data acquisition unit 131 then acquires the medical data selected by the user. The medical data acquisition unit 131 transmits the acquired medical data to the inference process package acquisition unit 132 and/or the processing unit 135, and the processing proceeds to step S22. The medical data acquired by the medical data acquisition unit 131 in step S21 is not limited to the above-described one. The medical data acquisition unit 131 may directly acquire the medical data of a process target, or the medical data selected by the user may be transmitted by an external apparatus.

In step S22, the inference process package acquisition unit 132 communicates with the data server 120, and presents, to the user, a list of the inference process packages in which the inference process by the inference model stored in the data server 120 and the preprocess and the post-process accompanying the inference process gathered into packages. The inference process package acquisition unit 132 then acquires the inference process package selected by the user. The inference process package may be selected by referring to a table and a selection condition set according to information about the medical data acquired by the medical data acquisition unit 131. The inference process package acquisition unit 132 selects the inference process package to be acquired based on the medical data. The selection may be realized by a different apparatus, such as an information terminal. The inference process package acquisition unit 132 acquires the inference process package to be applied to the medical data, transmits the acquired inference process package to the determination unit 133, and the processing proceeds to step S23. The inference process package may be set in advance and stored in the data server 120. Alternatively, a process selected by the user may be stored as a package. Furthermore, a single or a plurality of processes included in the inference process package may also be appropriately changed, corrected, or selected by a user.

In step S23, the determination unit 133 determines whether two or more inference process packages are acquired. In a case where two or more inference process packages are acquired by the inference process package acquisition unit 132 (YES in step S23), the processing proceeds to step S24. In a case where the acquired inference process package is one (NO in step S23), the processing proceeds to step S25.

In step S24, the determination unit 133 determines a process common to two or more inference process packages in the plurality of acquired inference process packages. A determination flow performed by the determination unit 133 is described below as an example.

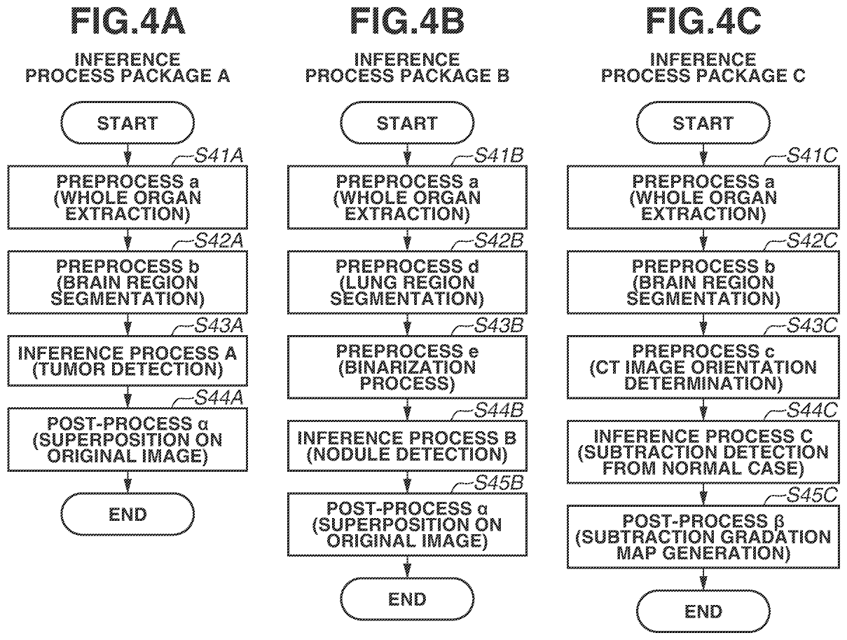

For example, it is assumed that the inference process package acquisition unit 132 acquires three inference process packages, e.g., an inference process package A, an inference process package B, and an inference process package C. Configurations of processes in the three inference process packages are described with reference to FIGS. 4A to 4C. The processes included in the inference process packages and procedures are not limited to the following example. In FIG. 4A, the inference process package A includes a preprocess a (a whole organ extraction process), a preprocess b (a brain region segmentation process), an inference process A (a tumor detection process), and a post-process a (a superposition process on an original image). The inference process package B illustrated in FIG. 4B includes the preprocess a (the whole organ extraction process), a preprocess d (a lung region segmentation process), a preprocess e (a binarization process), an inference process B (a pulmonary nodule detection process), and the post-process α.

The inference process package C illustrated in FIG. 4C includes the preprocess a (the whole organ extraction process), the preprocess b (the brain region segmentation process), a preprocess c (a CT image orientation determination process), an inference process C (a subtraction detection process from a normal case), and a post-process β (a subtraction gradation map generation process).

A specific example of the determination executed by the determination unit 133 with respect to the above-described inference process packages is described.

It is assumed that the single or the plurality of processes included in the respective inference process packages illustrated in FIGS. 4A to 4C are executed in the order described in the respective configurations. In step S24, the determination unit 133 first determines whether there is a common process in a first stage process of each inference process package. In the examples in FIGS. 4A to 4C, the preprocess a (steps S41A, S41B, and S41C) is the process common to the inference process packages A, B, and C. The determination unit 133 then determines whether a next process is common to each inference process package that includes the common first stage process (a first common process). In the examples illustrated in FIGS. 4A to 4C, the preprocess b (steps S42A and S42C) is a process common to the inference process packages A and C, and therefore the determination unit 133 determines the process performed in steps S42A and S42C as a second common process. The determination unit 133 repeats similar determination until there are no inference process packages that include a common process. In other words, the determination unit 133 determines the common process from the processes executed before the inference process among the single or the plurality of processes included in the inference process packages. In the above-described examples, a next process is not common to the inference process packages A and C, and therefore the determination is terminated at this step. In the determination of the common process by the determination unit 133, one process is determined as a common process, if the process is common to two or more inference process packages among the plurality of acquired inference process packages. Thus, it is not necessary to determine whether the process is common to all of the plurality of acquired inference process packages. In a case where the inference process package acquisition unit 132 acquires a plurality of inference process packages, the determination unit 133 determines a process common to two or more inference process packages among the processes included in the plurality of inference process packages. After the determination unit 133 performs determination for a common process, the determination unit 133 transmits the determination result and information about the inference process packages to the setting unit 134, and the processing proceeds to step S25.

In step S25, the setting unit 134 sets the processing procedure for specifying the order of processes to be applied to the medical data based on the information about the plurality of inference process packages acquired from the inference process package acquisition unit 132. The setting unit 134 sets the processing procedure such that the process common to the plurality of inference process packages which is determined by the determination unit 133 is applied to the medical data, and then other processes are executed. In other words, the setting unit 134 sets the processing procedure such that the process common to the plurality of inference process packages is applied to the medical data, and then the other processes included in the plurality of inference process packages are individually executed in each inference process package. The individual process is executed based on the processing result of the process common to the plurality of inference process packages.

In the examples in FIGS. 4A to 4C, the processing procedures set by the setting unit 134 are as follows. First, the preprocess a (steps S41A, S41B, and S41C) is applied, and then the preprocess b (steps S42A and S42C) is applied to the medical data. Thereafter, in the inference process packages A and C, remaining processes (steps S43A and S44A in the inference process package A, and steps S43C, S44C, and S45C in the inference process package C) are applied to the medical data to which the preprocesses a and b are applied. In the inference process package B, remaining processes (steps S42B, S43B, S44B, and S45B) are applied to the medical data to which the preprocess a is applied. The setting unit 134 sets the above-described processing procedures, and then outputs processing procedure information to the processing unit 135. The processing proceeds to step S26.

In step S26, the processing unit 135 acquires the medical data of the process target from the medical data acquisition unit 131. The processing unit 135 acquires the processing procedure that is set by the setting unit 134 and is applied to the medical data, and acquires a necessary inference model and processing information from the medical data acquisition unit 131. In a case where there is the process common to the plurality of inference process packages, the processing unit 135 first applies the common process to the medical data based on the processing procedure set by the setting unit 134 and temporarily stores the application result in the storage unit 136. The processing unit 135 executes the process common to the plurality of inference process packages determined by the determination unit 133, and then executes the remaining processes of each inference process package to the medical data to which the temporarily stored common processing result is applied. In a case where there is no common process, the processing unit 135 executes the process of each inference process package to the medical data acquired from the medical data acquisition unit 131 based on the processing procedure set by the setting unit 134. The processing unit 135 executes all processes included in the inference process package to be applied and then transmits the processing result to the storage unit 136. The processing unit 135 also transmits the processing result to the output unit 137.

In step S27, the storage unit 136 acquires the processing result of the processing unit 135, stores the processing result in the data server 120. The processing proceeds to step S28.

In step S28, the output unit 137 outputs the processing result transmitted from the processing unit 135 to the not-illustrated display unit. Alternatively, the output unit 137 outputs the processing result to an external apparatus. The output unit 137 also outputs the output result of the processing unit 135 in association with the inference process package by which the process is executed.

The information processing apparatus 130 executes the above-described process flow, and thereby the determination unit 133 can determine the process common to the plurality of processes included in the plurality of inference process packages acquired by the inference process package acquisition unit 132. Furthermore, the setting unit 134 sets the processing procedure based on the determination result of the determination unit 133, the processing unit 135 executes the process based on the set processing procedure, and thus the process can be executed to the medical data at an appropriate number of processing times. Any of the processes in the above-described flowchart may be executed by a different apparatus or may be omitted.

As described above, the information processing apparatus 130 according to the present example embodiment can execute the processes including the inference process at a minimum number of processing times even if a plurality of inference process packages to be applied to the medical data includes a common process. A plurality of inference processes can thereby be efficiently executed.

In the example described above, preprocesses to be applied to the medical data for executing the inference process has a common process. However, the information processing apparatus 130 executes a process in a similar manner to the above-described method, even in a case where preprocesses and the inference processes have a common process or a case where a part of the preprocesses, the inference processes, and the post-processes to be applied to the result of the inference processes have a common process.

An information processing system according to a second example embodiment is described. An inference process to be applied to medical data may determine whether to execute another inference process from a result of a certain inference process, such as determining a disease in a case where an abnormality is detected.

According to the present example embodiment, a case is described in which a process in an inference process package is completed, and then a process in another inference process package is additionally executed after confirming the processing result. In the following description, a configuration similar to that according to the first example embodiment is denoted by the same reference numeral, and the description thereof is omitted.

### (Determination Flow)

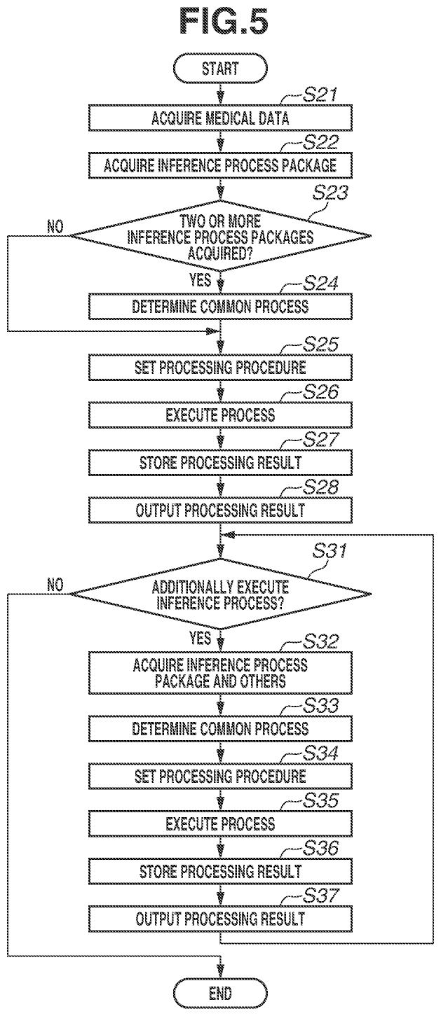

Processes executed by the information processing apparatus 130 according to the present example embodiment are described with reference to a flowchart illustrated in FIG. 5. According to the present example embodiment, the processes up to step S28 are similar to those executed in the first example embodiment, so that processes in step S31 and subsequent steps are described.

In step S31, the information processing apparatus 130 determines whether to additionally execute the inference process in response to a user instruction. In a case where the inference process is additionally executed (YES in step S31), the processing proceeds to step S32. In a case where the inference process is not additionally executed (NO in step S31), the processing ends.

In step S32, the inference process package acquisition unit 132 communicates with, for example, the data server 120, presents a list of inference process package information to the user, and acquires additional inference process package information in response to the selection by the user. An acquisition method of the inference process package by the inference process package acquisition unit 132 is not limited to the above-described method. For example, the inference process package acquisition unit 132 may select the inference process package by referring to a table according to a type of the medical data acquired by the medical data acquisition unit 131, or may store a condition in advance and select the inference process package satisfying the condition. The inference process package acquisition unit 132 also communicates with the data server 120 and acquires each processing result and processing information associated with the processing result. The inference process package acquisition unit 132 then outputs the acquired information to the determination unit 133. The processing proceeds to step S33.

In step S33, the determination unit 133 determines whether there is a common process between the processes included in the additionally acquired inference process package and the processes applied to each processing result acquired from the data server 120. A method for determining the common process is the same as that performed in step S24. The determination unit 133 transmits the determination result to the setting unit 134. The processing proceeds to step S34.

In step S34, the setting unit 134 sets the processing procedure to be applied to the medical data in a similar manner to that performed in step S25. Regarding the common process between the processed data stored in the data server 120 and the additionally acquired inference process package, the setting unit 134 sets the processing procedure such that the processing unit 135 substitutes the process by acquiring the processed medical data from the data server 120. In the following description, a substitute of the above-described process is referred to as a substitute process. The setting unit 134 sets the processing procedure, and then transmits information about the set processing procedure to the processing unit 135. The processing proceeds to step S35.

In step S35, the processing unit 135 acquires the processing procedure set by the setting unit 134. The processing unit 135 also acquires information about the necessary inference model and the process from the data server 120 via the medical data acquisition unit 131. In a case where the processing procedure includes the substitute process, the processing unit 135 acquires, from the data server 120, the processed medical data to be substituted via the medical data acquisition unit 131. The processing unit 135 executes the processes of the inference process package with respect to the processed medical data acquired from the data server 120 or the medical data not yet processed according to the processing procedure in a similar manner to that performed in step S26. The processing unit 135 then transmits the processing result to the storage unit 136 and the output unit 137. The processing proceeds to step S36.

In step S36, the storage unit 136 acquires the processing result of the processing unit 135, stores the processing result in the data server 120. The processing proceeds to step S37.

In step S37, the output unit 137 outputs the processing result of the medical data generated by the processing unit 135. The processing then proceeds to step S31.

As described above, the information processing apparatus 130 according to the present example embodiment can use the result of the common process already executed even in a case where the result of the inference process is confirmed and then another inference process is executed. The determination unit 133 can thereby determine the common process and efficiently execute the inference process even in a case where the inference process is additionally executed.

### <First Modification>

According to the above-described example embodiments, image data captured and obtained by the image capturing apparatus is described as an example of medical data, but the example embodiment of the present disclosure is not limited to image data. In addition to image data, the medical data may include waveform data of, for example, an electrocardiogram, text data of electronic medical record information, and any combination of these medical data. The medical data includes at least one of image data, waveform data, or text data. Further, the inference process package acquisition unit 132 may change the inference process package to be a target, according to an input data format.

The inference process package including the preprocess, the inference process, and the post-process is described as an example of the inference process package. However, the configuration of the inference process package is not limited to the above-described configuration. The configuration of the inference process package may include at least one inference process and may not include the preprocess and the post-process. A minimum configuration is an inference process package including only one inference process. In such a case, it is determined whether the inference process is a common process with respect to an inference process package that does not include the preprocess and executes the inference process first.

The inference process package acquisition unit 132 acquires the inference process package by a user selecting the inference process package to be applied to the medical data. However, the acquisition method of the inference process package is not limited to the above-described method. For example, a plurality of inference process packages may be automatically acquired using a machine learning model that has learned a combination of inference process packages to be acquired with respect to medical data as an input.

### <Second Modification>

According to the above-described example embodiments, the storage unit 136 stores all the medical data to which the process of the inference process package is applied, in the data server 120. However, the example embodiment of the present disclosure is not limited to the above-described one. For example, the storage unit 136 may cause the output unit 137 to output medical data to the input and output interfaces of the information processing apparatus 130, and a user may select the processing result to be stored in the data server 120 via the interface. Each processing result of the processing unit 135 may also be displayed with respect to the output interface of the information processing apparatus 130. The storage unit 136 may store a display method in outputting by the output unit 137 in association with each inference process package information stored in the data server 120. In a case where the output unit 137 outputs each processing result in the above-described case, the output unit 137 changes the display method of the processing result based on information about the display method associated with the inference process package information.

The output unit 137 can thereby change the display method of the processing result based on a process content of the inference process package, and thus a user can more easily confirm the processing result.

An information processing system according to a third example embodiment is now described. The information processing system according to the present example embodiment further includes an information terminal 740 communicable with an information processing apparatus 130 via the network in addition to the configuration of the above-described information processing system. A user can select an inference process package by operating the information terminal 740 to display an output result of the information processing apparatus 130 thereon.

The information terminal 740 further includes a selection unit 741 for selecting the inference process package and a display unit 742 for displaying an output from the information processing apparatus 130. Examples of the information terminal 740 include a personal computer (PC) terminal, a mobile phone such as a smartphone, a notebook terminal, and a tablet terminal.

The information processing apparatus 130 causes the display unit 742 in the information terminal 740 to output, via the network, the output from the output unit 137 described according to the above-described example embodiments. For example, the information processing apparatus 130 causes the display unit 742 in the information terminal 740 to display a list of the inference process packages, and a user may select the inference process package via the selection unit 741 in the information terminal 740.

The inference process package acquisition unit 132 in the information processing apparatus 130 acquires the inference process package in response to the selection of the inference process package by the selection unit 741.

The information processing apparatus 130 outputs the processing result to the information terminal 740, and the information terminal 740 causes the display unit 742 to display based on the received output. According to the present example embodiment, the information processing apparatus 130 can select an inference process package and confirm a processing result according to an optimized processing procedure, even in a case where the information processing apparatus 130 is configured with, for example, a cloud and an apparatus different from the information terminal 740 operated by a user.

The present disclosure can also be realized by executing the following process. Specifically, software (a program) that realizes functions of the above-described example embodiment is supplied to a system or an apparatus via a network or various storage media, and a computer (or e.g., a CPU and a micro processing unit (MPU)) of the system or the apparatus reads out the program and executes the process.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)?), a flash memory device, a memory card, and the like.

While the example embodiments are disclosed, it is to be understood that the invention is not limited to the disclosed example embodiments. The scope of the following claims is to be accorded the broadest interpretation so as to encompass all such modifications and equivalent structures and functions.

This application claims the benefit of Japanese Patent Application No. 2020-201484, filed Dec. 4, 2020, which is hereby incorporated by reference herein in its entirety.

What is claimed is:

1. An information processing apparatus comprising:
an inference process package acquisition unit configured to acquire an inference process package that is applied to medical data and includes a single or a plurality of processes including an inference process;
a determination unit configured to, in a case where a plurality of the inference process packages is acquired, determine a process common to greater than or equal to two inference process packages among processes included in the plurality of the inference process packages; and
a setting unit configured to set a procedure of the plurality of processes including the common process to be applied to the medical data based on a determination result of the determination unit.

2. The information processing apparatus according to claim 1, further comprising a medical data acquisition unit configured to acquire the medical data.

3. The information processing apparatus according to claim 2, further comprising a processing unit configured to execute a process on the medical data based on the procedure of the processes set by the setting unit.

4. The information processing apparatus according to claim 1, wherein the inference process package further includes a process accompanying the inference.

5. The information processing apparatus according to claim 1, wherein the setting unit sets a procedure to include another process included in the inference process package after the common process.

6. The information processing apparatus according to claim 1, wherein the determination unit determines the common process from processes to be applied before the inference process in the single or the plurality of processes included in the inference process package.

7. The information processing apparatus according to claim 1, wherein the inference process package acquisition unit acquires a plurality of inference process packages selected by a user.

8. The information processing apparatus according to claim 3, further comprising an output unit configured to output a result of processing performed by the processing unit and an inference process package in which the processing is performed in association with each other.

9. The information processing apparatus according to claim 1, wherein the medical data includes at least one of image data, waveform data, or text data.

10. The information processing apparatus according to claim 9, wherein the inference process package acquisition unit selects an inference process package to be acquired based on the medical data.

11. A method for processing information, the method comprising:
acquiring an inference process package that is applied to medical data and includes a single or a plurality of processes including an inference process;
determining, in a case where a plurality of the inference process packages is acquired, a process common to greater than or equal to two inference process packages among processes included in the plurality of the inference process packages; and
setting a procedure of the plurality of processes including the common process to be applied to the medical data based on a determination result.

12. A non-transitory storage medium storing a program for causing a computer to execute the method according to claim 11.

13. An information processing system including an information processing apparatus and an information terminal configured to communicate with the information processing apparatus via a network,
wherein the information processing apparatus includes:
an inference process package acquisition unit configured to acquire an inference process package that is applied to medical data and includes a single or a plurality of processes including an inference process;
a determination unit configured to, in a case where a plurality of the inference process packages is acquired, determine a process common to greater than or equal to two inference process packages among processes included in the plurality of the inference process packages; and
a setting unit configured to set a procedure of the plurality of processes including the common process to be applied to the medical data based on a determination result of the determination unit.

14. The information processing system according to claim 13,
wherein the information processing apparatus further includes:
a processing unit configured to execute a process on the medical data based on a set procedure of processes; and
an output unit configured to output a result of processing executed by the processing unit to the information terminal, and
wherein the information terminal further includes a display unit configured to perform display based on the output.

15. The information processing system according to claim 13,
wherein the information terminal further includes a selection unit configured to select an inference process package, and
wherein the inference process package acquisition unit acquires the inference process package in response to selection by the selection unit.
